(19) United States
(12) Patent Application Publication
(10) Pub. No.: US 2021/0000530 A1
(43) Pub. Date: Jan. 7, 2021

(54) CORDLESS SURGICAL DEVICE AND CONTROL METHOD

(71) Applicant: OLYMPUS CORPORATION, Tokyo (JP)

(72) Inventor: Minoru Kawasaki, Tokyo (JP)

(73) Assignee: OLYMPUS CORPORATION, Tokyo (JP)

(21) Appl. No.: 17/028,326

(22) Filed: Sep. 22, 2020

Related U.S. Application Data
(63) Continuation of application No. PCT/JP2018/012212, filed on Mar. 26, 2018.

(51) Int. Cl. A61B 18/12

(52) U.S. Cl. CPC A61B 18/1206; A61B 2018/00702

(57) ABSTRACT

A cordless surgical device includes a battery and a memory that stores data in which, with each of a plurality of energy output modes in which living tissue is treated by applying energy to the living tissue, a minimum power necessary to execute the energy output mode is associated, and a processor. The processor monitors a remaining power in the battery, selects an energy output mode in which the minimum power is less than the remaining power in the battery from among the energy output modes, and executes the selected energy output mode. The energy output modes include a normal output mode and an urgent output mode between which types of energy applied to the living tissue are the same. The minimum power corresponding to the normal output mode is higher than the minimum power corresponding to the urgent output mode.

This application is a continuation of International Application No. PCT/JP2018/012212, filed on Mar. 26, 2018, the entire contents of which are incorporated herein by reference.

### BACKGROUND

The present disclosure relates to a cordless surgical device and a control method.

A cordless surgical device that includes a battery and that treats a site to be treated (“subject site” below) in living tissue by applying ultrasound energy to the subject site has been known.

In the known cordless surgical device, when the charge in the battery is equal to or larger than a given threshold, it is possible to apply ultrasound energy to a subject site. On the other hand, in the cordless surgical device, when the charge in the battery is under the given threshold, application of ultrasound energy to the subject site is prohibited.

### SUMMARY

According to one aspect of the present disclosure, there is provided a cordless surgical device that includes a battery, a memory configured to store data including a value of a minimum power necessary to execute each of a plurality of energy output modes in which living tissue is treated by respectively applying a different type of energy to the living tissue, and a processor. The processor is configured to: (i) monitor a remaining power in the battery; (ii) select one energy output mode of the plurality of energy output modes based on the stored values of minimum power in which the minimum power is smaller than the remaining power in the battery; (iii) execute the selected energy output mode of the plurality of energy output modes, each energy output mode includes a normal output mode and an urgent output mode that each apply the same type of energy to the living tissue, the minimum power corresponding to the normal output mode being higher than the minimum power corresponding to the urgent output mode; (iv) determine, based on the stored data, whether the remaining power in the battery is less than the minimum power corresponding to the normal output mode when the normal output mode is executed; (v) preferentially select, as the energy output mode in which the minimum power is less than the remaining power in the battery among the plurality of energy output modes based on the stored data, the urgent output mode in which the type of energy is the same as the type of energy of the normal output mode over another energy output mode in which the type of energy is different from the type of energy of the normal output mode upon determining that the remaining power in the battery is less than the minimum power corresponding to the normal output mode; and (vi) execute the selected urgent output mode.

The above and other features, advantages and technical and industrial significance of this disclosure will be better understood by reading the following detailed description of present embodiments of the disclosure, when considered in connection with the accompanying drawings.

### BRIEF DESCRIPTION OF THE DRAWINGS

FIG. 1 is a diagram illustrating a cordless surgical device according to a first embodiment;

FIG. 2 is a block diagram illustrating the cordless surgical device;

FIG. 3 is a diagram illustrating an association information that is stored in a memory;

FIG. 4 is a flowchart illustrating a control method that is executed by a processor;

FIG. 5 is a block diagram illustrating a cordless surgical device according to a second embodiment; and

FIG. 6 is a diagram illustrating association information that is stored in a memory.

### DETAILED DESCRIPTION

With reference to the drawings, modes for carrying out the disclosure (“embodiments” below) will be described below. The embodiments described below do not limit the disclosure. In the drawings, the same components are denoted by the same reference numbers.

Configuration of Cordless Surgical Device

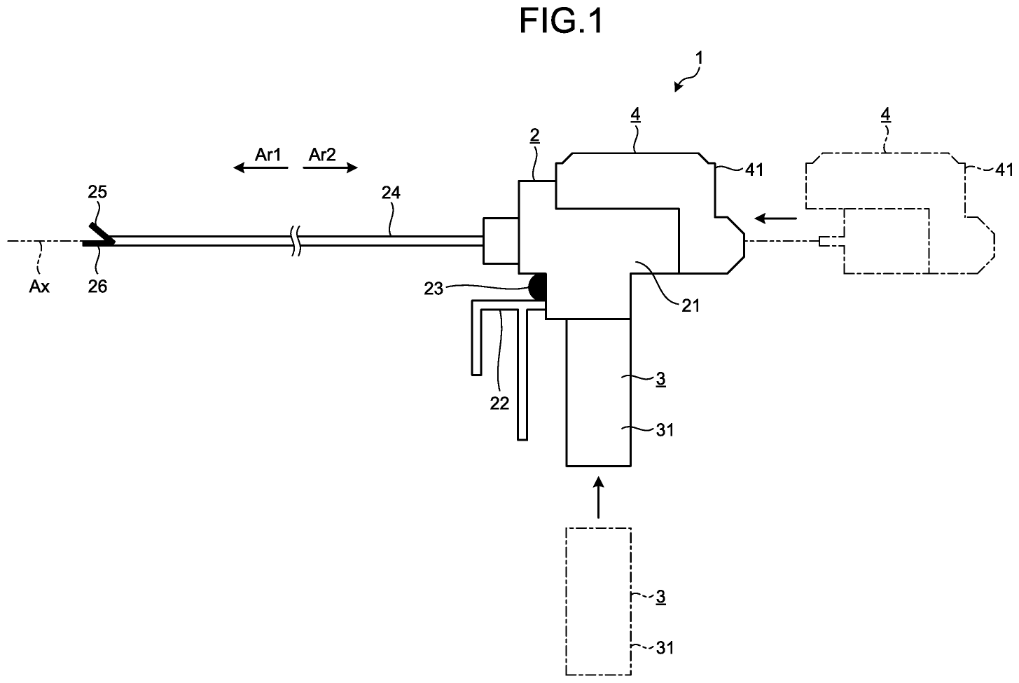

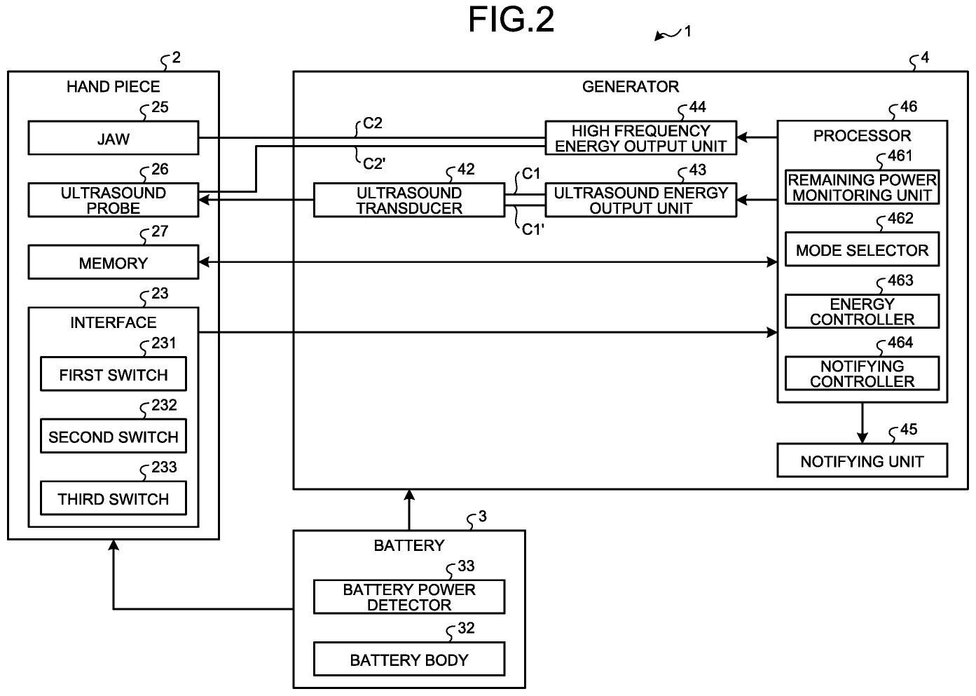

FIG. 1 is a block diagram illustrating a cordless surgical device 1 according to a first embodiment. FIG. 2 is a block diagram illustrating the cordless surgical device 1.

The cordless surgical device 1 treats a region to be treated (“subject site” below) in living tissue by applying energy to the subject site. Treatments executable by the cordless surgical device 1 according to the first embodiment includes three treatments: a first treatment of simultaneously coagulating and cutting the subject site, a second treatment of only cutting the subject site, and a third treatment of only sealing the subject site. In the first embodiment, the cordless surgical device 1 is a medical treatment tool using a bolt-clamped Langevin type transducer (BLT) for treating the subject site with the medical treatment tool penetrating the abdominal wall. As illustrated in FIG. 1 or FIG. 2, the cordless surgical device 1 includes a hand piece 2, a battery 3, and a generator 4.

Configuration of Hand Piece

As illustrated in FIG. 1 or FIG. 2, the hand piece 2 includes a holder case 21 (FIG. 1), an operation knob 22 (FIG. 1), an interface 23, a sheath 24 (FIG. 1), a jaw 25, an ultrasound probe 26, and a memory 27 (FIG. 2).

The holder case 21 supports the whole cordless surgical device 1.

The operation knob 22 is movably attached to the holder case 21 and receives an open/close operation performed by a practitioner.

The interface 23 is arranged in a state of being exposed to the outside of the holder case 21 and receives operations of setting first to third energy output modes performed by the practitioner. As illustrated in FIG. 2, the interface 23 includes first to third switches 231 to 233.

The first switch 231 receives an operation of setting a first energy output mode that is performed by the practitioner. The first switch 231 outputs an operation signal corresponding to the setting operation to a processor 46 that configures the generator 4.

The second switch 232 receives an operation of setting a second energy output mode that is performed by the practitioner. The second switch 232 outputs an operation signal corresponding to the setting operation to the processor 46.

The third switch 233 receives an operation of setting a third energy output mode that is performed by the practitioner. The third switch 233 outputs an operation signal corresponding to the setting operation to the processor 46.

The sheath 24 has a cylindrical shape. The center axis of the sheath 24 is referred to as a center axis Ax (FIG. 1) below. One side along the center axis Ax is referred to as a distal end side Ar1 (FIG. 1) and the other side will be referred to as a proximal end side Ar2 (FIG. 1). Part of the sheath 24 on the proximal end side Ar2 is inserted from the distal end side Ar1 of the holder case 21 into the holder case 21 and is thus attached to the holder case 21.

The jaw 25 is rotatably attached to an end of the sheath 24 on the distal end side Ar1 and grasps a subject site between the jaw 25 and part of the ultrasound probe 26 on the distal end side Ar1. In the holder case 21 and the sheath 24 described above, an open-close mechanism (not illustrated in the drawings) that causes the jaw 25 to open or close with respect to the part of the ultrasound probe 26 on the distal end side Ar1 according to an operation of open or close the operation knob 22 performed by the practitioner is arranged.

The ultrasound probe 26 has an elongated shape extending linearly along the center axis Ax and, as illustrated in FIG. 1, is inserted into the sheath 24 with the part on the distal end side Ar1 protruding to the outside. An end of the ultrasound probe 26 on the proximal end side Ar2 is connected to an ultrasound transducer 42 (FIG. 2) configuring the generator 4 via a horn (not illustrated in the drawings). The ultrasound probe 26 transmits ultrasound vibrations that are generated by the ultrasound transducer 42 and are transmitted through the horn (not illustrated in the drawings) from the end of the ultrasound probe 26 on the proximal end side Ar2 to the end on the distal end side Ar1 and applies the ultrasound vibrations to the subject site from the end on the distal end side Ar1, thereby treating the subject site.

The memory 27 stores programs (including a control program according to the disclosure) to be executed by the processor 46 and information necessary for processing performed by the processor 46. The following association information may be exemplified as the information necessary for the processing performed by the processor 46.

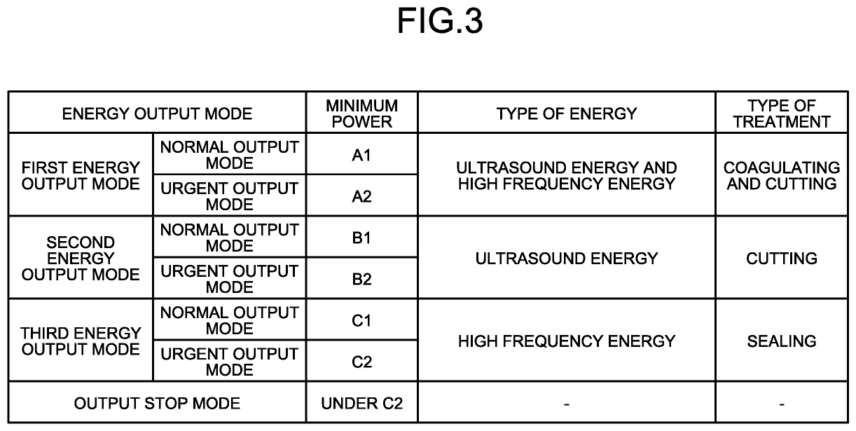

FIG. 3 is a diagram illustrating the association information that is stored in the memory 27.

As illustrated in FIG. 3, the association information is data in which, with each of the multiple output modes in which a subject site is treated by applying energy to the subject site, a minimum power necessary to execute the output mode, a type of energy, and a type of treatment are associated. The energy output modes include the first to third output modes and an output stop mode.

Specifically, the first output mode is an energy output mode in which a subject site is coagulated and cut by applying ultrasound energy and high frequency energy to the subject site. “Applying ultrasound energy to the subject site” means applying ultrasound vibrations to the subject site. “Applying high frequency energy to the subject site” means applying a high frequency current to the subject site. With the first energy output mode, as represented in FIG. 3, “ultrasound energy and high frequency energy” are associated as the type of energy and “coagulating and cutting” corresponding to the first treatment are associated as the type of treatment. The first energy output mode includes a normal output mode of driving at relatively high power and an urgent output mode of driving at relatively low power. With the normal output mode, as represented in FIG. 3, “A1” is associated as the minimum power. With the urgent output mode, “A2” is associated as the minimum power. The minimum power A1 is a power higher than the minimum power A2.

The second energy output mode is an energy output mode in which a subject site is cut by applying ultrasound energy to the subject site. With the second energy output mode, as represented in FIG. 3, “ultrasound energy” is associated as the type of energy and “cutting” corresponding to the second treatment is associated as the type of treatment. The second energy output mode includes a normal output mode of driving at relatively high power and an urgent output mode of driving at relatively low power. With the normal output mode, as represented in FIG. 3, “B1” is associated as the minimum power. With the urgent output mode, “B2” is associated as the minimum power. The minimum power B1 is a power lower than the minimum power A2.

The third energy output mode is an energy output mode in which a subject site is sealed by applying high frequency energy to the subject site. With the third energy output mode, as represented in FIG. 3, “high frequency energy” is associated as the type of energy and “sealing” corresponding to the third treatment is associated as the type of treatment. The third energy output mode includes a normal output mode of driving at relatively high power and an urgent output mode of driving at relatively low power. With the normal output mode, as represented in FIG. 3, “C1” is associated as the minimum power. With the urgent output mode, “C2” is associated as the minimum power. The minimum power C1 is a power higher than the minimum power C2 and lower than the minimum power B2.

The relationship among the minimum powers A1, A2, B1, B2, C1 and C2 is summarized into a relationship of A1>A2>B1>B2>C1>C2.

The output stop mode is an energy output mode in which applying energy to a subject site is stopped. With the output stop mode, as represented in FIG. 3, “under C2” is associated as the minimum power.

Configuration of Battery

As illustrated in FIG. 1 or FIG. 2, the battery 3 includes a battery case 31 (FIG. 1), a battery body 32 (FIG. 2) and a battery power detector 33 (FIG. 2).

The battery body 32 and the battery power detector 33 are housed in the battery case 31 and, as illustrated in FIG. 1, the battery case 31 is detachably connected to the holder case 21.

The battery body 32 is a secondary battery. With the battery case 31 being connected to the holder case 21, the battery body 32 electrically connects to at least the processor 46 among the interface 23, the memory 27 and an ultrasound energy output unit 43, a high frequency energy output unit 44, a notifying unit 45, and the processor 46 that configure the generator 4 and supplies power to at least the battery power detector 33.

The battery power detector 33 is an integrated circuit (IC) that detects the remaining power in the battery body 32. With the battery case 31 being connected to the holder case 21, the battery power detector 33 electrically connects to the processor 46 and outputs a signal corresponding to the detected remaining power to the processor 46.

Configuration of Generator

As illustrated in FIG. 1 or FIG. 2, the generator 4 includes a generator case 41 (FIG. 1), the ultrasound transducer 42 (FIG. 2), the ultrasound energy output unit 43 (FIG. 2), the high frequency energy output unit 44 (FIG. 2), the notifying unit 45 (FIG. 2), and the processor 46 (FIG. 2).

The generator case 41 supports the ultrasound transducer 42, the ultrasound energy output unit 43, the high frequency energy output unit 44, the notifying unit 45, and the processor 46 and, as illustrated in FIG. 1, is detachably connected to the holder case 21.

Under the control of the processor 46, the ultrasound transducer 42 generates ultrasound vibrations. In the first embodiment, the ultrasound vibrations are vertical vibrations in a direction along the center axis Ax. Although not illustrated in the drawings, the ultrasound transducer 42 is a BLT including a plurality of piezoelectric elements that are laminated along the center axis Ax. With the generator case 41 being connected to the holder case 21, the ultrasound transducer 42 connects to the part of the ultrasound probe 26 on the proximal end side Ar2 via the horn (not illustrated in the drawings).

The ultrasound energy output unit 43 is electrically connected to the ultrasound transducer 42 via first current paths C1 and C1′ in a pair (FIG. 2). Under the control of the processor 46, the ultrasound energy output unit 43 supplies alternating power to the ultrasound transducer 42 via the first current paths C1 and C1′ in a pair. Accordingly, the ultrasound transducer 42 generates ultrasound vibrations.

With the generator case 41 being connected to the holder case 21, the high frequency energy output unit 44 electrically connects to the jaw 25 and the ultrasound probe 26 via second current paths C2 and C2′ (FIG. 2). Under the control of the processor 46, the high frequency energy output unit 44 supplies a high frequency current between the jaw 25 and the ultrasound probe 26 via the second current paths C2 and C2′ in a pair. The high frequency current thus flows into the subject site that is grasped by the jaw 25 and the part of the ultrasound probe 26 on a distal end side Ar1. In other words, the jaw 25 and the ultrasound probe 26 also function as high frequency electrodes.

Under the control of the processor 46, the notifying unit 45 notifies given information. For example, a light emitting diode (LED) that notifies the given information by lighting, blinking, or color of lighting, a display device that displays the given information, or a speaker that outputs the given information by sound can be exemplified as the notifying unit 45.

The processor 46 is, for example, a central processing unit (CPU) or a field-programmable gate array (FPGA) and electrically connects to the interface 23 and the memory 27 with the generator case 41 being connected to the holder case 21. According to the program that is stored in the memory 27, the processor 46 controls operations over the cordless surgical device 1. As illustrated in FIG. 2, the processor 46 includes a remaining power monitoring unit 461, a mode selector 462, an energy controller 463, and a notifying controller 464.

The remaining power monitoring unit 461 monitors the remaining power in the battery body 32 according to a signal that is output from the battery power detector 33. The remaining power monitoring unit 461 compares the remaining power with each of the minimum powers A1, A2, B1, B2, C1 and C2 in the association information that is stored in the memory 27.

The mode selector 462 selects any one of the energy output modes according to an operation signal that is output from the interface 23 and the result of comparison performed by the remaining power monitoring unit 461.

The energy controller 463 controls operations of at least any one of the ultrasound energy output unit 43 and the high frequency energy output unit 44 and executes the energy output mode that is selected by the mode selector 462.

The notifying controller 464 causes the notifying unit 45 to notify the given information according to the result of comparison performed by the remaining power monitoring unit 461.

Control Method Performed by Processor

A control method performed by the above-described processor 46 will be described.

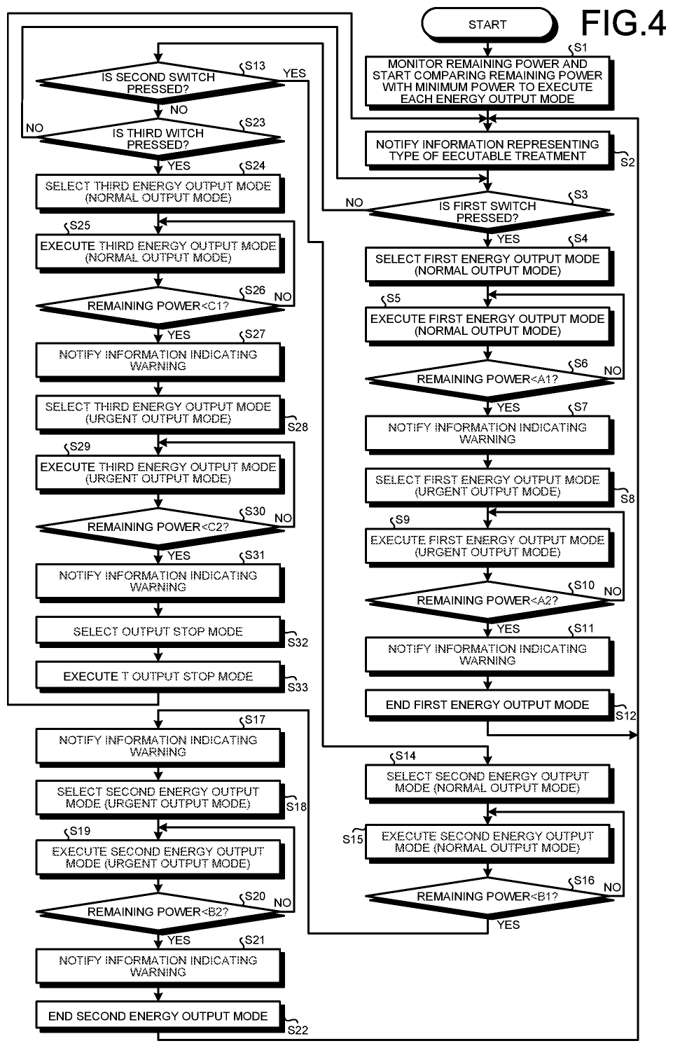

FIG. 4 is a flowchart illustrating the control method performed by the processor 46.

First of all, the remaining power monitoring unit 461 monitors the remaining power in the battery body 32 according to a signal that is output from the battery power detector 33 and starts comparing the remaining power with each of the minimum powers in the association information that is stored in the memory 27 (step S1).

After step S1, according to the result of comparison performed by the remaining power monitoring unit 461, the notifying controller 464 causes the notifying unit 45 to notify information representing the type of executable treatment (step S2).

For example, when the remaining power monitoring unit 461 determines that the remaining power is equal to or larger than the minimum power A1, at step S2, the notifying controller 464 refers to the association information and causes the notifying unit 45 to notify the following information as information representing the type of executable treatment. Specifically, the notifying controller 464 causes the notifying unit 45 to notify information representing “coagulating and cutting” that is associated with the first energy output mode corresponding to the minimum power A1, information representing “cutting” that is associated with the second energy output mode corresponding to the minimum power B1 smaller than the minimum power A1, and information representing “sealing” that is associated with the third energy output mode corresponding to the minimum power C1 smaller than the minimum power B1. For convenience of description, the notification made by the notifying unit 45 is referred to as first notification below. The first notification allows the practitioner to recognize that all the first to third treatments are executable.

For example, when the remaining power monitoring unit 461 determines that the remaining power is under the minimum power A1 and is equal to or larger than the minimum power B1, at step S2, the notifying controller 464 refers to the association information and causes the notifying unit 45 to notify the following information as information indicating the type of executable treatment. Specifically, the notifying controller 464 causes the notifying unit 45 to notify information representing “cutting” that is associated with the second energy output mode corresponding to the minimum power B1 and information representing “sealing” that is associated with the third energy output mode corresponding to the minimum power C1 smaller than the minimum power B1. For the convenience of description, the notification made by the notifying unit 45 is referred to as second notification below. The second notification allows the practitioner to recognize that, among the first to third treatments, the second and third treatments are executable.

When the remaining power monitoring unit 461 determines that the remaining power is under the minimum power B1 and is equal to or larger than the minimum power C1, at step S2, the notifying controller 464 refers to the association information and causes the notifying unit 45 to notify the following information as information indicating the type of executable treatment. Specifically, the notifying controller 464 causes the notifying unit 45 to notify information representing “sealing” that is associated with the third energy output mode corresponding to the minimum power C1. For the convenience of description, the notification made by the notifying unit 45 is referred to as third notification below. The third notification allows the practitioner to recognize that, among the first to third treatments, only the third treatment is executable.

For example, when the remaining power monitoring unit 461 determines that the remaining power is under the minimum power C1, at step S2, the notifying controller 464 causes the notifying unit 45 to notify information representing that all the first to third treatments are not executable. For the convenience of description, the notification made by the notifying unit 45 is referred to as fourth notification below. The fourth notification allows the practitioner to recognize that all the first to third treatments are not executable.

After recognizing the executable treatment from the notification at step S2, the practitioner causes the part of the cordless surgical device 1 on the distal end side Ar1 to penetrate through the abdominal wall using a trocar, or the like, and then inserts the part into the abdominal cavity. The practitioner performs an operation to close and open the operation knob 22 to grasp the subject site with the jaw 25 and the part of the ultrasound probe 26 on the distal end side Ar1. Thereafter, when performing the first treatment, the practitioner presses the first switch 231 (YES at step S3). When the first switch 231 is pressed (YES at step S3), the mode selector 462 selects the normal output mode in the first energy output mode (step S4).

After step S4, the energy controller 463 executes the normal output mode in the first energy output mode that is selected by the mode selector 462 (step S5). Specifically, the energy controller 463 controls operations of the high frequency energy output unit 44 and supplies a relatively higher high frequency current between the jaw 25 and the ultrasound probe 26 via the second current paths C2 and C2′ in a pair. Approximately simultaneously with the supply of the high frequency current between the jaw 25 and the ultrasound probe 26, the energy controller 463 controls operations of the ultrasound energy output unit 43 and supplies relatively high alternating power to the ultrasound transducer 42 via the first current paths C1 and C1′ in a pair. In other words, the energy controller 463 causes the ultrasound transducer 42 to generate ultrasound vibrations. The vertical vibrations of the ultrasound probe 26 causes the part of the ultrasound probe 26 on the distal end side Ar1 to vibrate, for example, at an amplitude of 80 μm. In other words, the high frequency current flows into the subject site that is grasped with the jaw 25 and the part of the ultrasound probe 26 on the distal end side Ar1 and accordingly Joule heat is generated. The vertical vibrations of the ultrasound probe 26 generates frictional heat between the part of the ultrasound probe 26 on the distal end side Ar1 and the subject site. Coagulating and cutting serving as the first treatment on the subject site are then started.

After step S5, the remaining power monitoring unit 461 keeps monitoring whether the remaining power in the battery body 32 is under the minimum power A1 (step S6).

When it is determined that the remaining power is equal to or larger than the minimum power A1 (NO at step S6), the energy controller 463 continues execution of the normal output mode (step S5).

On the other hand, when it is determined that the remaining power is under the minimum power A1 (YES at step S6), the notifying controller 464 causes the notifying unit 45 to notify information that indicates a warning and that represents that it is not possible to continue execution of the normal output mode (step S7).

After step S7, the mode selector 462 refers to the association information that is stored in the memory 27 and selects the urgent output mode in the first energy output mode that is associated with the minimum power A2 under the minimum power A1 (step S8).

After step S8, the energy controller 463 executes the urgent output mode in the first energy output mode that is selected by the mode selector 462 (step S9). Specifically, the energy controller 463 controls operations of the high frequency energy output unit 44 and lowers the high frequency current supplied between the jaw 25 and the ultrasound probe 26 via the second current paths C2 and C2′ in a pair, or makes intermittent outputs. The energy controller 463 controls operations of the ultrasound energy output unit 43 and lowers the alternating power that is supplied to the ultrasound transducer 42 via the first current paths C1 and C1′ in a pair. Accordingly, the part of the ultrasound probe 26 on the distal end side Ar1 vibrates, for example, at an amplitude of 50 μm that is smaller than the amplitude in the normal output mode. In other words, even when the energy output mode switches from the normal output mode to the urgent output mode, coagulating and cutting the subject site is continued while the outputs of the ultrasound energy and the high frequency energy applied to the subject site lower.

After step S9, the remaining power monitoring unit 461 keeps monitoring whether the remaining power in the battery body 32 is under the minimum power A2 (step S10).

When it is determined that the remaining power is equal to or larger than the minimum power A2 (NO at step S10), the energy controller 463 continues execution of the urgent output mode (step S9).

On the other hand, when it is determined that the remaining power is under the minimum power A2 (YES at step S10), the notifying controller 464 causes the notifying unit 45 to notify information that indicates a warning and that represents that it is not possible to continue execution of the urgent output mode, that is, the first energy output mode (step S11).

After step S11, the energy controller 463 ends executing the first energy output mode (step S12). Thereafter, the processor 46 returns to step S2. In other words, after step S12, the notifying unit 45 makes the second notification at step S2.

After recognizing the executable treatment from the notification at step S2, the practitioner causes the part of the cordless surgical device 1 on the distal end side Ar1 to penetrate through the abdominal wall using a trocar, or the like, and inserts the part into the abdominal cavity. The practitioner performs an operation to close and open the operation knob 22 to grasp the subject site with the jaw 25 and the part of the ultrasound probe 26 on the distal end side Ar1. Thereafter, when performing the second treatment, the practitioner presses the second switch 232 (YES at step S13). When the second switch 232 is pressed (YES at step S13), the mode selector 462 selects the normal output mode in the second energy output mode (step S14).

After step S14, the energy controller 463 executes the normal output mode in the second energy output mode that is selected by the mode selector 462 (step S15). Specifically, the energy controller 463 controls operations of the ultrasound energy output unit 43 and supplies relatively high alternating power to the ultrasound transducer 42 via the first current paths C1 and C1′ in a pair. In other words, the energy controller 463 causes the ultrasound transducer 42 to generate ultrasound vibrations. The vertical vibrations of the ultrasound probe 26 causes the part of the ultrasound probe 26 on the distal end side Ar1 to vibrate, for example, at an amplitude of 80 μm. In other words, the vertical vibrations of the ultrasound probe 26 generate frictional heat between the subject part, which is grasped with the jaw 25 and the part of the ultrasound probe 26 on the distal end side Ar1, and the ultrasound probe 26. Cutting serving as the second treatment on the subject site is then started.

After step S15, the remaining power monitoring unit 461 keeps monitoring whether the remaining power in the battery body 32 is under the minimum power B1 (step S16).

When it is determined that the remaining power is equal to or larger than the minimum power B1 (NO at step S16), the energy controller 463 continues execution of the normal output mode (step S15).

On the other hand, when it is determined that the remaining power is under the minimum power B1 (YES at step S6), the notifying controller 464 causes the notifying unit 45 to notify information that indicates a warning and that represents that it is not possible to continue execution of the normal output mode (step S17).

After step S17, the mode selector 462 refers to the association information that is stored in the memory 27 and selects the urgent output mode in the second energy output mode that is associated with the minimum power B2 under the minimum power B1 (step S18).

After step S18, the energy controller 463 executes the urgent output mode in the second energy output mode that is selected by the mode selector 462 (step S19). Specifically, the energy controller 463 controls operations of the ultrasound energy output unit 43 and lowers the alternating power that is supplied to the ultrasound transducer 42 via the first current paths C1 and C1′ in a pair. Accordingly, the part of the ultrasound probe 26 on the distal end side Ar1 vibrates, for example, at an amplitude of 50 μm that is smaller than the amplitude in the normal mode. In other words, even when the energy output mode switches from the normal output mode to the urgent output mode, cutting the subject site is continued while the output of the ultrasound energy applied to the subject site lowers.

After step S19, the remaining power monitoring unit 461 keeps monitoring whether the remaining power in the battery body 32 is under the minimum power B2 (step S20).

When it is determined that the remaining power is equal to or larger than the minimum power B2 (NO at step S20), the energy controller 463 continues execution of the urgent output mode (step S19).

On the other hand, when it is determined that the remaining power is under the minimum power B2 (YES at step S20), the notifying controller 464 causes the notifying unit 45 to notify information that indicates a warning and that represents that it is not possible to continue execution of the urgent output mode, that is, the second energy output mode (step S21).

After step S21, the energy controller 463 ends executing the second energy output mode (step S22). Thereafter, the processor 46 returns to step S2. In other words, after step S22, the notifying unit 45 makes the third notification at step S2.

After recognizing the executable treatment from the notification at step S2, the practitioner causes the part of the cordless surgical device 1 on the distal end side Ar1 to penetrate through the abdominal wall using a trocar, or the like, and inserts the part into the abdominal cavity. The practitioner performs an operation to close and open the operation knob 22 to grasp the subject site with the jaw 25 and the part of the ultrasound probe 26 on the distal end side Ar1. Thereafter, when performing the third treatment, the practitioner presses the third switch 233 (YES at step S23). When the third switch 233 is pressed (YES at step S23), the mode selector 462 selects the normal output mode in the third energy output mode (step S24).

After step S24, the energy controller 463 executes the normal output mode in the third energy output mode that is selected by the mode selector 462 (step S25). Specifically, the energy controller 463 controls operations of the high frequency energy output unit 44 and supplies a relatively higher high frequency current between the jaw 25 and the ultrasound probe 26 via the second current paths C2 and C2′ in a pair. In other words, the high frequency current flows into the subject site that is grasped with the jaw 25 and the part of the ultrasound probe 26 on the distal end side Ar1 and accordingly Joule heat is generated. Sealing serving as the third treatment on the subject site is then started.

After step S25, the remaining power monitoring unit 461 keeps monitoring whether the remaining power in the battery body 32 is under the minimum power C1 (step S26).

When it is determined that the remaining power is equal to or larger than the minimum power C1 or larger (NO at step S26), the energy controller 463 continues execution of the normal output mode (step S25).

On the other hand, when it is determined that the remaining power is under the minimum power C1 (NO at step S26), the notifying controller 464 causes the notifying unit 45 to notify information that indicates a warning and that represents that it is not possible to continue execution of the normal output mode (step S27).

After step S27, the mode selector 462 refers to the association information that is stored in the memory 27 and selects the urgent output mode in the third energy output mode that is associated with the minimum power C2 under the minimum power C1 (step S28).

After step S28, the energy controller 463 executes the urgent output mode in the third energy output mode that is selected by the mode selector 462 (step S29). Specifically, the energy controller 463 controls operations of the high frequency energy output unit 44 and lowers the high frequency current supplied between the jaw 25 and the ultrasound probe 26 via the second current paths C2 and C2′ in a pair, or makes intermittent outputs. In other words, even when the energy output mode is switched from the normal output mode to the urgent output mode, sealing the subject site is continued while the output of the high frequency energy applied to the subject site lowers.

After step S29, the remaining power monitoring unit 461 keeps monitoring whether the remaining power in the battery body 32 is under the minimum power C2 (step S30).

When it is determined that the remaining power is equal to or larger than the minimum power C2 (NO at step S30), the energy controller 463 continues execution of the urgent output mode (step S29).

On the other hand, when it is determined that the remaining power is under the minimum power C2 (YES at step S30), the notifying controller 464 causes the notifying unit 45 to notify information representing that it is not possible to execute the urgent output mode, that is, the third energy output mode (step S31).

After step S31, the mode selector 462 refers to the association information that is stored in the memory 27 and selects the output stop mode that is associated with being under the minimum power C2 (step S32).

After step S32, the energy controller 463 executes the output stop mode that is selected by the mode selector 462 (step S33). Specifically, the energy controller 463 stops driving the high frequency energy output unit 44 and stops applying the high frequency energy to the subject site. Thereafter, the processor 46 returns to step S2. In other words, after step S33, the notifying unit 45 makes the fourth notification at step S2.

According to the first embodiment described above, the following effect is achieved.

The cordless surgical device 1 according to the first embodiment monitors the remaining power in the battery body 32 and, based on the association information that is stored in the memory 27, selects an energy output mode whose corresponding in which the minimum power is smaller than the remaining power in the battery body 32 from among the energy output modes. The cordless surgical device 1 then executes the selected energy output mode.

Particularly, in the first embodiment, when the normal output mode is being executed, the cordless surgical device 1 determines based on the association information whether the remaining power in the battery body 32 is under the minimum power necessary to execute the normal output mode. When it is determined that the remaining power is under the minimum power, the cordless surgical device 1 preferentially selects the urgent output mode in which the type of energy is the same as that of the normal output mode over another energy output mode in which the type of energy is different from that of the normal output mode. The cordless surgical device 1 then executes the selected urgent output mode.

Thus, in the cordless surgical device 1, even when the remaining power in the battery body 32 is under the minimum power necessary to execute the normal output mode while the subject site is being treated by executing the normal output mode, the normal output mode is switched to the urgent output mode and accordingly it is possible to continue the treatment on the subject site. In other words, the treatment on the subject site is not stopped midway. Thus, according to the cordless surgical device 1 according to the first embodiment, it is possible to increase user-friendliness.

In the cordless surgical device 1 according to the first embodiment, when the remaining power in the battery body 32 is under the minimum power necessary to execute the normal output mode while the subject site is being treated by executing the normal output mode, information indicating a warning is notified.

This allows the practitioner to recognize that the energy output mode has switched from the normal output mode to the urgent output mode during the treatment on the subject site and deal with the switching as appropriate.

In the cordless surgical device 1 according to the first embodiment, information representing the type of treatment corresponding to the energy output mode in which the minimum power is smaller than the remaining power in the battery body 32 among the energy output modes is notified based on the association information.

This allows the practitioner to recognize the type of executable treatment before treating the subject site and deal with the treatment as appropriate.

A second embodiment will be described below.

In the following description, the same components as those of the first embodiment are denoted with the same reference numbers and detailed description thereof will be omitted or simplified.

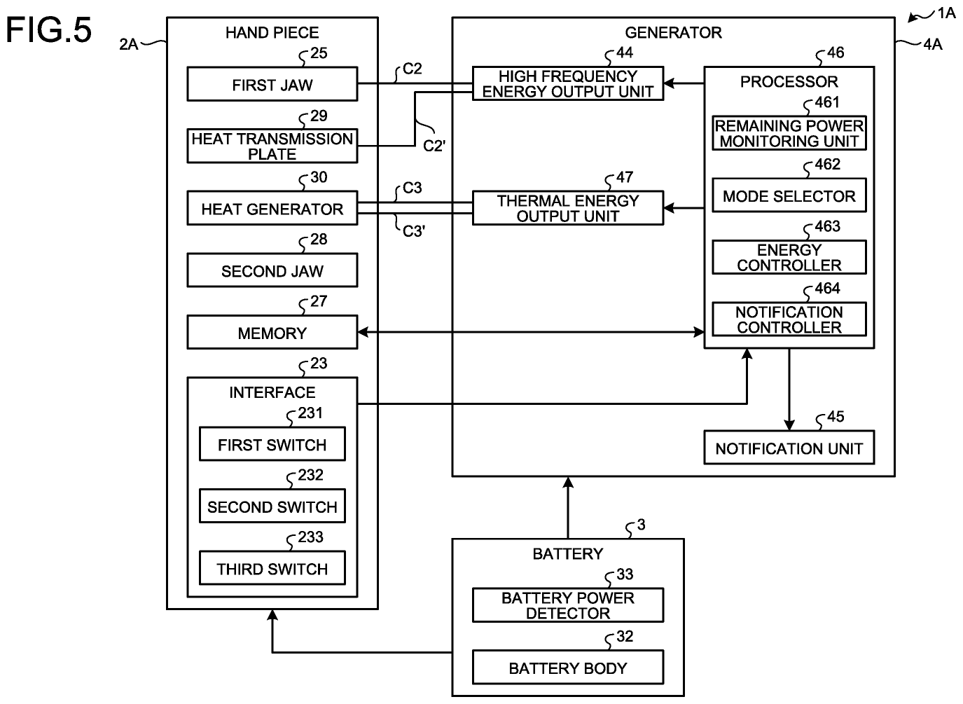

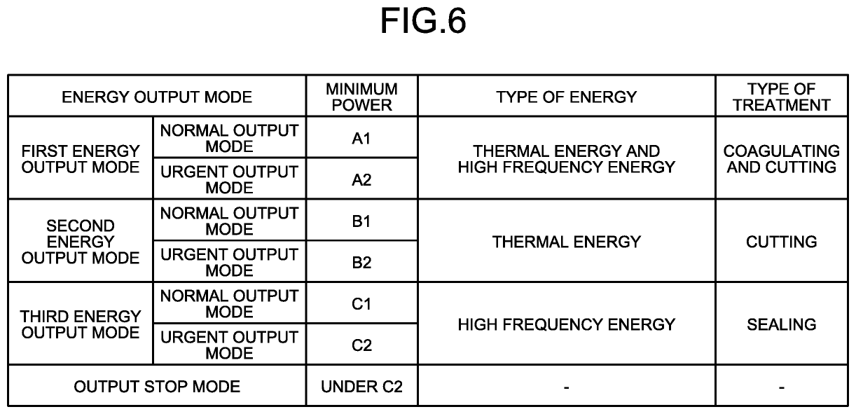

FIG. 5 is a block diagram illustrating a cordless surgical device 1A according to the second embodiment. FIG. 6 is a diagram illustrating association information that is stored in the memory 27 according to the second embodiment.

In the cordless surgical device 1 according to the first embodiment, a subject site is treated by applying ultrasound energy and high frequency energy to the subject site.

On the other hand, in the cordless surgical device LA according to the second embodiment, a subject site is treated by applying thermal energy and high frequency energy to the subject site.

Specifically, in a hand piece 2A according to the second embodiment, as illustrated in FIG. 5, instead of the ultrasound probe 26, a jaw 28, a heat transmission plate 29, and a heat generator 30 are employed against the hand piece 2 described in the first embodiment described above. In order to distinguishing the jaw 25 and the jaw 28 from each other, the jaw 25 is referred to as the first jaw 25 and the jaw 28 is referred to as the second jaw 28 below.

The second jaw 28 being opposed to the first jaw 25 is fixed to an end of the sheath 24 on the distal end side Ar1. The heat generator 30 and the heat transmission plate 29 are laminated on a surface of the second jaw 28 that is opposed to the first jaw 25 in the order they appear in this sentence. In other words, according to an operation of opening and closing the first jaw 25, the subject site is grasped between the first jaw 25 and the heat transmission plate 29.

The heat transmission plate 29 is, for example, a copper thin plate. The heat transmission plate 29 transmits heat from the heat generator 30 to the subject site. The heat transmission plate 29 electrically connects to the high frequency energy output unit 44 via the second current path C2′ (FIG. 5). Under the control of the processor 46, the high frequency energy output unit 44 supplies a high frequency current between the first jaw 25 and the heat transmission plate 29 via the second current path C2 and C2′ in a pair. Accordingly, the high frequency current flows into the subject site that is grasped between the first jaw 25 and the heat transmission plate 29. In other words, the heat transmission plate 29 also functions as a high frequency electrode.

The heat generator 30 is, for example, a sheet heater. Although not specifically illustrated, the heat generator 30 is obtained by forming an electric resistance pattern on a sheet substrate that is formed of an insulated material, such as polyimide, by vapor deposition, or the like.

The electric resistance pattern is, for example, formed along a U-shape following the outer circumferential shape of the heat generator 30. With the generator case 41 being connected to the holder case 21, both ends of the electric resistance pattern electrically connect to a thermal energy output unit 47 (FIG. 5) configuring a generator 4A via third current paths C3 and C3′ in a pair. Power is supplied from the thermal energy output unit 47 to the electric resistance pattern and accordingly the electric resistance pattern generates heat.

In association with the change of the type of energy to be applied to the subject site, the association information that is stored in the memory 27 is also changed.

Specifically, a first energy output mode according to the second embodiment is an energy output mode in which a subject site is coagulated and cut by applying thermal energy and high frequency energy to the subject site. “Applying thermal energy to the subject site” means applying heat from the heat generator 30 to the subject site via the heat transmission plate 29. With the first energy output mode, as represented in FIG. 6, “thermal energy and high frequency energy” are associated as the type of energy and “coagulating and cutting” corresponding to a first treatment are associated as the type of treatment. The first energy output mode includes, as in the above-described first embodiment, a normal output mode of driving at relatively high power and an urgent output mode of driving at relatively low power. With the normal output mode and the urgent output mode, as in the case of the above-described first embodiment, minimum powers A1 and A2 are associated, respectively.

A second energy output mode according to the second embodiment is an energy output mode in which a subject site is cut by applying thermal energy to the subject site. With the second energy output mode, as represented in FIG. 6, “thermal energy” is associated as the type of energy and “cutting” corresponding to a second treatment is associated as the type of treatment. The second energy output mode includes, as in the above-described first embodiment, a normal output mode of driving at relatively high power and an urgent output mode of driving at relatively low power. With the normal output mode and the urgent output mode, as in the above-described first embodiment, minimum powers B1 and B2 are associated, respectively.

Note that, as represented in FIG. 6, the third energy output mode and the output stop mode are the same as those of the above-described first embodiment.

As illustrated in FIG. 5, in the generator 4A according to the second embodiment, instead of the ultrasound energy output unit 43, the thermal energy output unit 47 is employed against the generator 4 described in the above-described first embodiment.

Under the control of the processor 46, the thermal energy output unit 47 supplies power to the electronic resistance pattern configuring the heat generator 30 via the third current paths C3 and C3′ in a pair. Accordingly, the electronic resistance pattern generates heat. The heat of the electronic resistance pattern is applied from the heat transmission plate 9 to the subject site that is grasped between the first jaw 25 and the heat transmission plate 29.

According to the control method performed by the processor 46 according to the second embodiment, when applying thermal energy to a subject site at steps S5 and S15, the processor 46 supplies a relatively high power to the electronic resistance pattern configuring the heat generator 30 via the third current paths C3 and C3′ in a pair and heats the subject site at a relatively high target temperature. At steps S9 and S19, the processor 46 supplies a relatively low power to the electronic resistance pattern via the third current paths C3 and C3′ in a pair and heats the subject site at a relatively low target temperature in the normal output mode.

Even the configuration in which thermal energy and high frequency energy are applied to a subject site as in the above-described second embodiment realizes the same effect as that of the above-described first embodiment.

Modes for carrying out the disclosure have been described; however, the disclosure should not be limited by only the above-described first and second embodiments.

The first and second embodiments respectively employ the configuration in which ultrasound energy and high frequency energy are applied to a subject site and the configuration in which thermal energy and high frequency energy are applied to a subject site; however, embodiments are not limited thereto. For example, a configuration in which ultrasound energy and thermal energy are applied to a subject site may be employed. For example, a configuration in which only any one of ultrasound energy, high frequency energy and thermal energy is applied to a subject site may be employed. In this case, energy output modes according to the disclosure includes a normal output mode and an urgent output mode in which the same energy is applied to a subject site but the power for driving differs between the modes.

In the above-described first and second embodiments, the association information is stored in the memory 27; however, embodiments are not limited thereto. For example, a configuration in which the association information is incorporated into the control program may be employed.

In the above-described first and second embodiments, the positions in which the memory 27 and the processor 46 are arranged are not limited to those illustrated in the above-described first and second embodiments, and the memory 27 and the processor 46 may be arranged in other positions. For example, the memory 27 may be arranged in the generators 4 and 4A. The processor 46 may be arranged in the hand pieces 2 and 2A.

The above-described first and second embodiments employ, as the energy output modes according to the disclosure, the six energy output modes of the first to third energy output modes each including the normal output mode and the urgent output mode; however, the embodiments are not limited thereto, and at least two energy output modes are preferably set. For example, a configuration in which any one of a normal output mode and an urgent output mode is not set may be employed. For example, a configuration in which the second energy output mode among the first to third energy output modes is not set may be employed.

In the above-described first and second embodiments, switching from the normal output mode to the urgent output mode continues the treatment on the subject site; however, embodiments are not limited thereto. For example, a configuration in which a treatment on a subject site is continued by switching from the first energy output mode to the second energy output mode or to the third energy output mode or by switching from the second energy output mode to the third energy output mode may be employed. In other words, during coagulating and cutting serving as the first treatment or cutting serving as the second treatment, when the remaining power in the battery body 32 lowers and execution of the first and second energy output modes cannot be continued, at least sealing serving as the third treatment is preferably continued.

The flow representing the control method performed by the processor 46 is not limited to the process order in the flowchart (FIG. 4) described in the above-described first and second embodiments and may be changed within a range causing no inconsistency.

According to the cordless surgical device, the control method, and the control program according to the disclosure, it is possible to increase user-friendless.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A cordless surgical device comprising:
a battery;
a memory configured to store data including a value of a minimum power necessary to execute each of a plurality of energy output modes in which living tissue is treated by respectively applying a different type of energy to the living tissue; and
a processor configured to:
monitor a remaining power in the battery;
select one energy output mode of the plurality of energy output modes based on the stored values of minimum power in which the minimum power is smaller than the remaining power in the battery;
execute the selected energy output mode of the plurality of energy output modes, each energy output mode includes a normal output mode and an urgent output mode that each apply the same type of energy to the living tissue, the minimum power corresponding to the normal output mode being higher than the minimum power corresponding to the urgent output mode;
determine, based on the stored data, whether the remaining power in the battery is less than the minimum power corresponding to the normal output mode when the normal output mode is executed;
preferentially select, as the energy output mode in which the minimum power is less than the remaining power in the battery among the plurality of energy output modes based on the stored data, the urgent output mode in which the type of energy is the same as the type of energy of the normal output mode over another energy output mode in which the type of energy is different from the type of energy of the normal output mode upon determining that the remaining power in the battery is less than the minimum power corresponding to the normal output mode; and
execute the selected urgent output mode.

2. The cordless surgical device according to claim 1, wherein the processor is configured to:
determine, based on the stored data, whether the remaining power in the battery is less than the minimum power corresponding to the energy output mode at a current time when any one of the plurality of energy output modes is executed,
select the energy output mode in which the minimum power is less than the remaining power in the battery from among the energy output modes upon determining that the remaining power in the battery is less than the minimum power corresponding to the energy output mode at the current time based on the stored data, and
execute the selected energy output mode.

3. The cordless surgical device according to claim 2, wherein the processor is configured to continue executing the energy output mode at the current time upon determining that the remaining power in the battery is greater than the minimum power corresponding to the energy output mode at the current time.

4. The cordless surgical device according to claim 1, wherein the plurality of energy output modes include an output stop mode in which outputting the energy is stopped.

5. The cordless surgical device according to claim 1, further comprising a hand piece to which the battery is detachably attached, the memory being located in the hand piece.

6. The cordless surgical device according to claim 5, further comprising a generator detachably attached to the hand piece and configured to generate ultrasound vibrations, the processor being located in the generator.

7. The cordless surgical device according to claim 1, wherein the plurality of energy output modes include at least two of:
a first energy output mode in which ultrasound energy and high frequency energy are simultaneously applied to the living tissue;
a second energy output mode in which only ultrasound energy is applied to the living tissue; and
a third energy output mode in which only high frequency energy is applied to the living tissue.

8. The cordless surgical device according to claim 1, wherein the plurality of energy output modes include at least two of:
a first energy output mode in which high frequency energy and thermal energy are simultaneously applied to the living tissue;
a second energy output mode in which only thermal energy is applied to the living tissue; and
a third energy output mode in which only high frequency energy is applied to the living tissue.

9. The cordless surgical device according to claim 1, further comprising an interface configured to receive an operation of setting any one of the plurality of energy output modes,
wherein the processor is configured to select any one of the plurality of energy output modes according to the operation on the interface.

10. The cordless surgical device according to claim 1, wherein the processor is configured to:
determine, based on the stored data, whether the remaining power in the battery is less than the minimum power corresponding to the energy output mode at the current time when any one of the plurality of energy output modes is executed, and
cause a notifying unit to notify information indicating a warning upon determining that the remaining power in the battery is less than the minimum power corresponding to the energy output mode at the current time.

11. The cordless surgical device according to claim 1, wherein:
the stored data for each of the plurality of energy output includes a type of treatment on the living tissue in the respective energy output mode, and
the processor is configured to, based on the stored data, cause a notifying unit to notify information representing the type of treatment corresponding to the energy output mode in which the minimum power is less than the remaining power in the battery from among the plurality of energy output modes.

12. A control method executed by a processor of a cordless surgical device, the method comprising:
monitoring a remaining power in a battery;
selecting, from among a plurality of energy output modes in which living tissue is treated by applying energy to the living tissue, one energy output mode of the plurality of energy output modes based on the stored values of minimum power in which a minimum power necessary to execute the selected energy output mode is smaller than the remaining power in the battery;
executing the selected energy output mode of the plurality of energy output modes, each energy output mode includes a normal output mode and an urgent output mode that each apply the same type of energy to the living tissue, the minimum power corresponding to the normal output mode being higher than the minimum power corresponding to the urgent output mode;
determining, based on the stored data, whether the remaining power in the battery is less than the minimum power corresponding to the normal output mode when the normal output mode is executed;
preferentially selecting, as the energy output mode in which the minimum power is less than the remaining power in the battery among the plurality of energy output modes based on the stored data, the urgent output mode in which the type of energy is the same as the type of energy of the normal output mode over another energy output mode in which the type of energy is different from that of the normal output mode upon determining that the remaining power in the battery is less than the minimum power corresponding to the normal output mode; and
executing the selected urgent output mode.
